(19) United States
(12) Patent Application Publication
(10) Pub. No.: US 2026/0096906 A1
(43) Pub. Date: Apr. 9, 2026

(54) BONE RASP AND GRAFT DELIVERY SHEATH AND RELATED METHODS

(71) Applicant: Medos International Sàrl, Le Locle (CH)

(72) Inventors: Pulak Ray, Newark, DE (US); William McLaughlin, Boston, MA (US); Thomas J. Runco, Providence, RI (US); Randel Betz, JR., Wilmington, RI (US)

(21) Appl. No.: 19/349,725

(22) Filed: Oct. 3, 2025

Related U.S. Application Data
(60) Provisional application No. 63/702,943, filed on Oct. 3, 2024.

(51) Int. Cl. A61F 2/46; A61B 17/16

(52) U.S. Cl. CPC A61F 2/4601; A61B 17/1671; A61F 2002/4635

(57) ABSTRACT

Surgical systems and methods for preparing multiple vertebra levels for spinal fusion are disclosed herein. An example method can include inserting a delivery sheath through a first incision formed proximate to a first pedicle screw, as well as advancing the delivery sheath toward one or more additional pedicle screws disposed in bone. The method can further include inserting an instrument with a cutting surface into the delivery sheath and passing the instrument over the bone in which the one or more additional pedicle screws are disposed to decorticate the bone with the cutting surface. The method can also include removing the instrument from the sheath, as well as passing at least one of a bone graft or a biologic material through the sheath to deliver the at least one of the bone graft or the biologic material to a surface of the bone exposed by the decorticating of the bone

### CROSS-REFERENCE TO RELATED APPLICATIONS

This application claims the benefit of U.S. Provisional Application No. 63/702,943, entitled “BONE RASP AND GRAFT DELIVERY SHEATH,” filed on Oct. 3, 2024. The entire contents of this application are hereby incorporated by reference in their entirety.

### FIELD

The present disclosure relates to systems, devices, and methods for preparing a bone surface, and more particularly relates to preparing one or more levels of spinal vertebra for fusion.

### BACKGROUND

Minimally invasive surgical procedures often require access to internal tissue. Specifically, orthopedic or neurologic surgical procedures, such as spinal surgeries, can require working channel through a small incision in a patient's tissue to access the vertebrae and/or intervertebral discs. Certain steps of a spinal surgical procedure can be difficult to perform through a small incision. For example, in a spinal fusion procedure where multiple levels of vertebrae are fused together, it can be difficult to decorticate and apply of bone graft material to the multiple levels of vertebrae through a small incision. No reliable methods or instrument designs exist for the effective rasping and preparation of the posterior spinal elements through a Minimally Invasive or Mini-Open incision. To accomplish single-level or multi-level bone preparation for posterior spinal fusion, the Minimally Invasive Surgery (MIS) incision size would need to be enlarged to provide access using current instrumentation. Further, with existing instruments, only a small segment of the spine could be rasped through an individual small incision. Additionally, there is not a reliable means for delivering biologic bone graft or bone-graft substitute in a controlled and targeted manner through an MIS incision, to the posterior spinal elements and onto the desired fusion area, e.g., in the “gutter” adjacent to the spinal rod location.

Accordingly, improved devices, systems, and methods are needed to prepare multiple levels of vertebrae through a minimally invasive incision.

### SUMMARY

The instruments, systems, and methods disclosed herein can allow for multiple spinal levels to be prepared for fusion by providing a rasping instrument configured to decorticate a length of bone surface through a small incision and a sheath which controls the manner and location of decortication and also the bone graft material application. The instruments disclosed herein can include a rasp intended for use in Minimally-Invasive Surgery (MIS procedures). The rasp or cutting instrument can include a long and thin member having teeth on the bottom surface that are intended to promote bleeding along the posterolateral “gutters” of the spine, adjacent to the location of a spinal rod construct. The rasped instrument can be specially designed to include a handle extending at an angle from the rasp (e.g., roughly perpendicularly, for an L-shaped instrument). The special design can allow the rasp instrument to be used in a minimally-invasive manner, while still allowing the surgeon to prepare the bone over multiple levels.

The instruments disclosed herein can also include an angled or L-shaped guiding sheath that can cover and nest with the rasp instrument, which can help to guide and constrain the motion of the rasp and to separate it from surrounding soft tissue. The bottom surface of the sheath can be concave, like a half-pipe, allowing it to nest with the rasp component. This elongated concave area can allow the elongated rasp to move in a back-and-forth manner while remaining against the bone surface and separated from the surrounding soft tissue.

The sheath also can provide a conduit for the controlled insertion and delivery of biologic material, such as ViviGen® (a cellular allograft for repair or reconstruction of musculoskeletal defects), within the area where the rasp was used to prepare the bone. After the rasping portion is complete, the rasp can be removed. The sheath can remain in place, providing a targeted pathway for the bone graft to be delivered along the posterior spine, adjacent to the spinal implant construct. It can prevent the bone graft material from migrating away from the fusion area during delivery and further helps to control the position and motion of the applicator tip. Further, the rasp instrument can be used to hold graft material in the desired location while the sheath is separated and removed from the surgical site. The instruments and methods disclosed herein therefore provide both a novel design and also a method for meeting the clinical requirements for treating the patient (e.g., with a spinal fusion).

Various embodiments and variations of the design are contemplated, including an adjustable handle and various geometries to help with its MIS use. Other embodiments can include a rasped surface that is flat or rounded, an elongated shape that is straight or curved, and/or a handle that is adjustable to different angles to facilitate insertion and rasping through an MIS/Mini-Open incision. In some embodiments, the handle can be removable to allow for different rasp and/or handle configurations according to surgeon preference and clinical requirements. Additional designs and/or features can be included to help control the positioning of the bone graft applicator tip. Examples can include an additional instrument or component to deliver the bone-graft delivery, and/or to function as an additional applicator tip and/or conduit for the targeted delivery of the bone graft material.

In one aspect, a method of preparing a bone for surgery can include inserting a delivery sheath through a first incision formed proximate to a first pedicle screw disposed in bone, as well as advancing the delivery sheath toward one or more additional pedicle screws disposed in bone. The method can further include inserting an instrument into the delivery sheath through the first incision, the instrument having an elongate shaft with a cutting surface formed thereon, as well as passing the instrument over the bone in which the one or more additional pedicle screws are disposed to decorticate the bone with the cutting surface. The method can also include removing the instrument from the sheath, as well as passing at least one of a bone graft or a biologic material through the sheath to deliver the at least one of the bone graft or the biologic material to a surface of the bone exposed by the decorticating of the bone.

Any of a variety of alternative or additional steps can be included and are considered within the scope of the present disclosure. For example, in some embodiments the method can further include palpating a head of a second pedicle screw of the one or more additional pedicle screws using a finger through a second incision formed proximate to the second pedicle screw, and advancing the delivery sheath toward the second pedicle screw disposed in bone can further include contacting the finger at the head of the second pedicle screw that was palpated.

In certain embodiments, the one or more additional pedicle screws can be a plurality of additional pedicle screws.

In some embodiments, advancing the delivery sheath toward one or more additional pedicle screws disposed in bone further can include advancing the delivery sheath to a distal-most pedicle screw of the one or more additional pedicle screws.

In certain embodiments, passing the instrument over the bone in which the one or more additional pedicle screws are disposed to decorticate the bone with the cutting surface can include angling the instrument in each of a cephalad direction and a caudal direction.

In some embodiments, passing the instrument over the bone in which the one or more additional pedicle screws are disposed to decorticate the bone with the cutting surface can include decorticating a dorsal aspect of an inferior articular process.

In certain embodiments, passing the instrument over the bone in which the one or more additional pedicle screws are disposed to decorticate the bone with the cutting surface can include decorticating a dorsal aspect of a transverse process.

In some embodiments, passing at least one of a bone graft or a biologic material through the sheath can include inserting a delivery instrument into the sheath to deliver the at least one of the bone graft or the biologic material to the surface of the bone exposed by the decorticating of the bone.

In certain embodiments, passing at least one of a bone graft or a biologic material through the sheath can include passing a bone graft through the sheath and, after the bone graft is delivered to the surface of the bone exposed by the decorticating of the bone. In addition, the method can further include reinserting the instrument into the sheath and contacting the delivered bone graft with the instrument. In some embodiments, the method can also include removing the sheath through the first incision so it is no longer disposed at the bone while keeping the instrument in contact with the bone graft.

In some embodiments, a distal portion of the sheath can extend at an angle relative to a proximal portion of the sheath and the proximal portion can extend out from the first incision after inserting the delivery sheath through the first incision.

In certain embodiments, a distal portion of the instrument can extend at an oblique angle relative to a proximal portion of the instrument and the proximal portion can extend out from the first incision after inserting the instrument into the delivery sheath.

In some embodiments, a longitudinal axis of a handle of the delivery sheath can form an oblique angle with a longitudinal axis of a handle of the instrument when the instrument is inserted into the delivery sheath.

In certain embodiments, passing the instrument over the bone in which the one or more additional pedicle screws are disposed to decorticate the bone with the cutting surface can include angling at least one of the instrument and the sheath in one of the medial or lateral directions and translating the instrument relative to the sheath along a longitudinal axis of a distal portion thereof.

In some embodiments, the method can further include adjusting a position of a handle of the instrument relative to the elongate shaft of the instrument.

In another aspect, a surgical system can include a cutting instrument that includes a handle and an elongate shaft, the elongate shaft having a proximal portion and a distal portion.

The distal portion can be disposed at an angle with respect to the proximal portion and the distal portion can have a cutting surface formed thereon. The system can further include a sheath having a handle and an elongate receiving portion that includes a channel extending from a proximal portion of the elongate receiving portion to a distal portion of the elongate receiving portion. The distal portion of the elongate receiving portion can be disposed at an angle with respect to the proximal portion of the elongate receiving portion, and the channel can be configured to receive the elongate shaft of the cutting instrument such that the cutting surface of the cutting instrument is exposed to be able to decorticate bone.

As with the methods described above, the systems disclosed herein can include any of a variety of additional or alternative features that are considered within the scope of the present disclosure. For example, in some embodiments the channel of the sheath can be open along a length thereof.

In some embodiments, a distal-most tip of the elongate receiving portion of the sheath can have an opening into the channel.

In certain embodiments, a distal-most tip of the elongate shaft of the cutting instrument can be configured to extend from the opening of the sheath when the distal portion of the elongate shaft is received within the distal portion of the elongate receiving portion of the sheath. In some embodiment, the distal-most tip of the elongate shaft can be bulleted.

In some embodiments, the distal portion of the elongate shaft and the distal portion of the elongate receiving portion of the sheath can be straight, each having a convex upper surface and a flat lower surface.

In certain embodiments, the distal portion of the elongate shaft and the distal portion of the elongate receiving portion of the sheath can be curved along a longitudinal axis thereof.

In some embodiments, the distal portion of the elongate receiving portion of the sheath can extend at a perpendicular angle relative to the proximal portion of the elongate receiving portion of the sheath.

In certain embodiments, the proximal portion of the elongate shaft of the cutting instrument can extend at an oblique angle relative to the distal portion of the elongate shaft of the cutting instrument.

In some embodiments, the handle can be fixed relative to the elongate shaft and a longitudinal axis of the handle can be coaxial with a longitudinal axis of the proximal portion of the elongate shaft.

In certain embodiments, the handle can be fixed relative to the elongate shaft and a longitudinal axis of the handle can form an oblique angle with a longitudinal axis of the proximal portion of the elongate shaft.

In some embodiments, a position of the handle can be adjusted relative to the elongate shaft.

In certain embodiments, the cutting surface can include a plurality of teeth.

In some embodiments, the distal portion of the elongate receiving portion of the sheath can be configured to receive an instrument for depositing bone graft.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF THE DRAWINGS

The aspects and embodiments of the present disclosure can be more fully understood from the following detailed description taken in conjunction with the accompanying drawings, in which:

FIG. 1 is a side view of one embodiment of a system including a cutting or rasp instrument and sheath in accordance with the present disclosure;

FIG. 2A is a side view of the cutting or rasp instrument of FIG. 1;

FIG. 2B is a side view of the sheath of FIG. 1;

FIG. 3 is a side view of another embodiment of a system including a sheath and a cutting or rasp instrument disposed therein;

FIG. 4 is a bottom perspective view of the assembly of FIG. 3;

FIG. 5A is a side view of another embodiment of a system including a sheath and a cutting or rasp instrument having an adjustable handle;

FIG. 5B is a top view of the system of FIG. 5A;

FIG. 5C is a cross-sectional view of the system of FIG. 5A;

FIG. 5D is a bottom view of the system of FIG. 5A;

FIG. 6A is perspective view of another embodiment of a sheath having an elongated distal portion;

FIG. 6B is perspective view of another embodiment of a sheath having an elongated distal portion;

6C is perspective view of another embodiment of a sheath having a shortened distal portion;

FIG. 6D is perspective view of another embodiment of a sheath having a shortened distal portion;

FIG. 7A is perspective view of another embodiment of a cutting or rasp instrument having an elongated distal portion;

FIG. 7B is perspective view of another embodiment of a cutting or rasp instrument having an elongated distal portion;

FIG. 7C is perspective view of another embodiment of a cutting or rasp instrument having a shortened distal portion;

FIG. 7D is perspective view of another embodiment of a cutting or rasp instrument having an shortened distal portion;

FIG. 8A is perspective view of a sheath having a curved distal portion;

FIG. 8B is a perspective view of a cutting or rasp instrument having a curved distal portion;

FIG. 9A is a posterior view of a spine with bone screws implanted into multiple levels of vertebrae and a rod extending through the bone screws;

FIG. 9B is a lateral view of one embodiment of a sheath being inserted in a minimally invasive incision;

FIG. 10A is a lateral view of a distal tip of the sheath of FIG. 9B moving from a proximal-most bone screw to an adjacent bone screw along a bone surface lateral to the bone screws;

FIG. 10B is a lateral view of the distal portion of the sheath of FIG. 9B continuing along the length of the spine while the proximal portion of the sheath extends from the minimally invasive incision;

FIG. 10C is a posterior view of a distal portion of the sheath of FIG. 9B positioned against a bone surface of multiple vertebral levels lateral to the bone screws and rod shown in FIG. 9A;

FIG. 10D is a lateral view of the sheath positioned as shown in FIG. 10C through a minimally invasive incision;

FIG. 11A is a lateral view of a cutting or rasp instrument inserted into the positioned sheath of FIG. 10D;

FIG. 11B is a lateral view of movement of the rasp instrument within the sheath of the FIG. 11A;

FIG. 11C is a posterior view of the assembly of FIG. 11B angled in the lateral direction relative to the spine;

FIG. 11D is a posterior view of the assembly of FIG. 11B angled in the medial direction relative to the spine;

FIG. 11E is a posterior view of vertebrae having bone surfaces decorticated by the assembly of FIG. 11B;

FIG. 12A is a lateral view of a bone graft delivery device inserted into a sheath positioned through a minimally invasive incision after removal of a cutting or rasp instrument;

FIG. 12B is a posterior view of a cannula of the bone graft delivery device of FIG. 12A against a spine;

FIG. 12C is a posterior view of the cannula of the bone graft delivery device of FIG. 12B retracting along the length of the spine;

FIG. 12D is a posterior view of the cannula of the bone graft delivery device of FIG. 12C further retracting along the length of the spine;

FIG. 13A is a lateral view of a cutting or rasp instrument reinserted into the sheath of FIG. 12A after the bone graft delivery device is removed; and

FIG. 13B is a lateral view illustrating removal of the cutting or rasp instrument and sheath of FIG. 13A.

### DETAILED DESCRIPTION

Certain example embodiments will now be described to provide an overall understanding of the principles of the structure, function, manufacture, and use of the devices, systems, and methods disclosed herein. One or more examples of these embodiments are illustrated in the accompanying drawings. The devices, systems, and methods specifically described herein and illustrated in the accompanying drawings are non-limiting embodiments. The features illustrated or described in connection with one embodiment may be combined with the features of other embodiments. Such modifications and variations are intended to be included within the scope of the present disclosure.

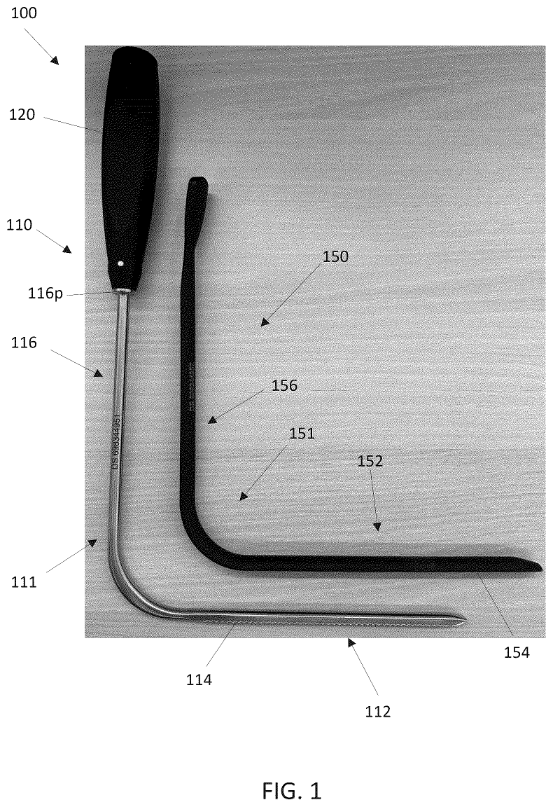

FIG. 1 illustrates one embodiment of a system 100 including a cutting or rasp instrument 110 and an associated sheath 150 for use in preparing spinal vertebrae for accepting bone graft during a spinal fusion procedure. The cutting or rasp instrument 110 can include an elongate shaft 111 having a distal portion 112 extending at an angle from a proximal portion 116. The shaft 111 can have a cutting surface 114 along the bottom or distal length of the distal portion 112 and a handle 120 coupled to the proximal end 116p. Similarly, the sheath 150 can include an elongate body 151 having a distal portion 152 extending at an angle relative to a proximal portion 156. The sheath 150 can further include a central channel 154 extending at least partially through the elongate body 151 that can receive the instrument 110 therethrough. The central channel 154 can be open along the bottom or distal length of the elongate body 151 to allow the cutting surface 114 of the instrument 110 to extend out from the distal portion 152 of the sheath 150.

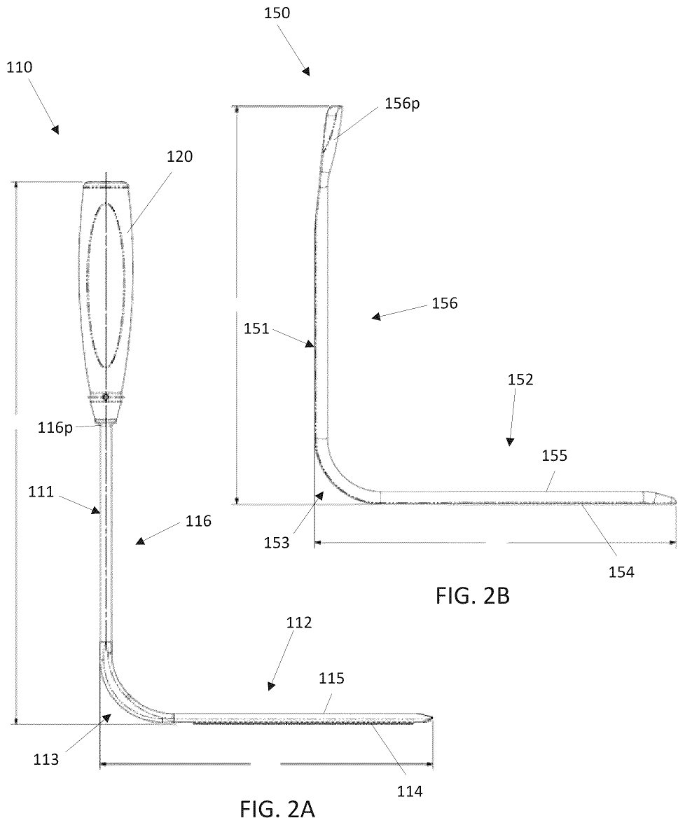

Referring to FIGS. 2A and 2B, the elongate shaft 111 of the rasp instrument 110 can be generally L-shaped. In other words, the distal portion 112 of instrument 110 can extend at a substantially perpendicular angle relative to the proximal portion 116. The elongate shaft 111 can further include a curved intermediate portion 113 between the distal portion 112 and the proximal portion 116. The distal portion 152 of the sheath can extend substantially perpendicular relative to the proximal portion 156 with an intermediate portion 153 between the distal portion 152 and the proximal portion 154. The illustrated angles of the rasp instrument 110 and sheath 150 are non-limiting. While the rasp instrument 110 and the sheath 150 are illustrated as having substantially perpendicular angles, there can be a difference in the angle between the rasp instrument 110 and the sheath 150 to facilitate nesting of the distal portion 112 of the rasp instrument 110 within the distal portion 152 of the sheath instrument. In some embodiments, the distal portion 152 of the sheath can extend at an oblique angle relative to the proximal portion 156 of the sheath and/or the distal portion 112 of the instrument 110 can extend at an oblique angle relative to the proximal portion 116. These angles can be the same or different for each of the sheath 150 and the instrument 110 to facilitate their cooperation as described herein.

In the illustrated embodiment, the proximal portion 116 of the rasp instrument 110 is cylindrical and the distal portion 112 is flattened to a shape having a convex upper surface 115 and a flat lower cutting surface 114. The sheath elongate body 151 can have a convex upper surface 155 matching the curvature of the convex upper surface 115 of the distal portion 112 of the instrument 110 such that the distal portion 152 of the sheath 150 can fully cover the upper surface 115 of the distal portion 112 of the instrument 110 leaving only the lower cutting surface 114 exposed. In some embodiments, the cutting surface 114 can be formed on a convex bottom surface of a generally cylindrical distal portion 112 of the instrument 110 such to facilitate rasping or cutting of curved spinal surfaces.

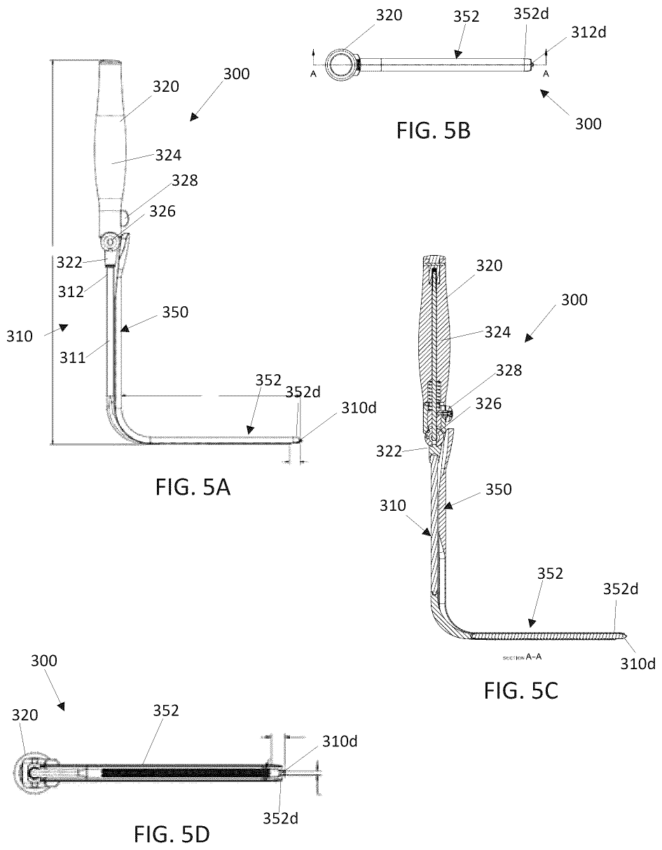

The handle 120 can be formed integrally with the proximal portion 116 of the instrument 110 or can be a separate component mated to the proximal end 116p of the instrument 110. In the present embodiment, the handle 120 is coupled to the proximal end 116p in a fixed position relative to the elongate shaft 111 and extends along a longitudinal axis of the proximal portion 116 of the elongate shaft 111. In some embodiments, as discussed with respect to FIG. 5A-5D, the handle 120 can have features to allow for adjustment or articulation of its position relative to the elongate shaft 111 or can be otherwise angled relative to the proximal portion 116 of the elongate shaft 111. In the illustrated embodiment, the channel 154 extends through the proximal portion 156 and distal portion 152 of the sheath 150 to allow nesting of both the proximal portion 116 and distal portion 112 of the rasp instrument 110 within the sheath 150. In some embodiments, the channel 154 extends through only the distal portion 152 or through the distal portion 152 and curved intermediate portion 153 to allow for nesting of the distal portion 112 of the rasp instrument within the distal portion 152 of the sheath while the proximal portion 116 is separated from the proximal portion 156 of the sheath and extending any of parallel or at an angle relative thereto. The proximal portion 156 of the sheath 150 can include a flattened proximal end 156p that provides clearance for the handle 120 of the instrument 110 when the instrument is received within the sheath 150 and the proximal portion 116 of the instrument 110 is held adjacent to the proximal portion 156 of the sheath 150.

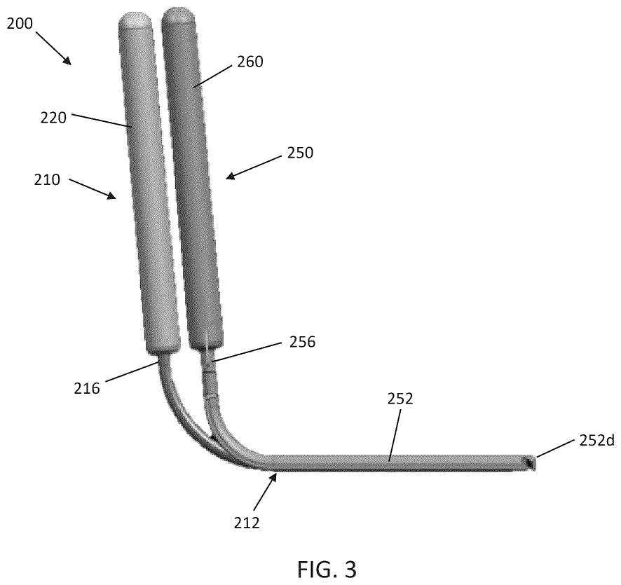

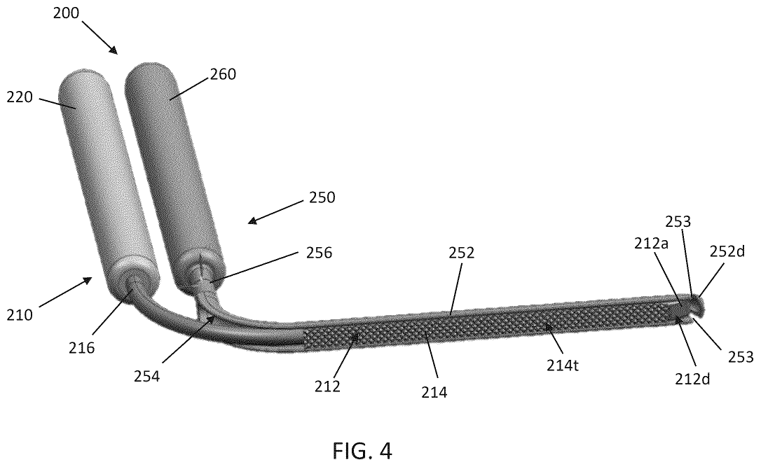

FIGS. 3 and 4 illustrate another embodiment of an assembly 200 including a rasp or cutting instrument 210 disposed within an associated sheath 250. In the illustrated embodiment, the proximal portion 256 of the sheath 250 is coupled to a handle 260, similar to the proximal portion 216 of the instrument 210. The instrument 210 can nest within the sheath 250 such that the distal portion 212 of the rasp 210 is received within the distal portion 252 of the sheath 250 and the proximal portion 216 of the rasp extends substantially parallel with the proximal portion 256 of the sheath. To allow clearance for the instrument handle 220 and the sheath handle 260, the distal portion 212 and the proximal portion 216 of the instrument can be at a larger angle than the distal portion 252 and the proximal portion 256 of the sheath. For example, the proximal portion 256 of the sheath 250 can extend at a substantially perpendicular angle relative to the distal portion 252 while the proximal portion 216 of the instrument 210 extends at an oblique angle relative to the distal portion 212. Alternatively or in addition, a larger curved intermediate portion between the portions 212, 216 can be utilized for the instrument 210 relative to the sheath 250 to provide separation between the handles 220, 260 when the instrument 210 and sheath 250 are nested together.

As shown in FIG. 4, the distal portion 212 of the instrument 210 can be received within the channel 254 of the distal portion 252 of the sheath 250 such that the cutting surface 214 is exposed from the open channel 254 to engage with a bone surface. The cutting surface 214 can include a plurality of teeth 214t extending therefrom. The distal tip 212d of the rasp instrument 210 can be wedged or include an angled surface 212a that is received within a rounded distal tip 252d of the sheath 250. The wedged tip 212d of the instrument 210 and rounded tip 252d of the sheath 250 can allow for smooth passage through soft tissue. The rounded tip 252d of the sheath can be partially separated from the distal portion 252 by two opposing cutouts 253 and at a distance from the distal tip 212d of the instrument 210 to allow flexing when the rounded tip 252d abuts against bone, an implant, or other component while moving through soft tissue.

FIG. 5A-5D illustrate various views of a third embodiment an assembly 300 including a sheath 350 and a rasp instrument 310 having an adjustable position or articulating handle 320. In the illustrated embodiment, a handle 320 is coupled to a proximal end 312 of the elongate shaft 311 of the instrument 310 such that it can be adjusted or articulated to various angled positions relative to a proximal portion of the elongate shaft. To facilitate such adjustment, the handle 320 can include a fixed distal end 322 coupled to the proximal end of the elongate shaft 311 and a proximal handle body 324 coupled to the fixed distal end 322 via pivot point 326. The proximal handle body 324 can pivot about the pivot point 326 to position a longitudinal axis of the proximal handle body 324 at various angles relative to the longitudinal axis of the of the elongate shaft 311. The adjustable handle 320 can further include a lock 328 to selectively lock and/or release the position of the handle 320 relative to the elongate shaft 111.

The distal end 352d of the sheath 350 can be open to allow a distal-most tip of the instrument 310 to extend out from the channel when received within the sheath. The distal-most tip 310d of the instrument 310 can be bulleted or curved to navigate through soft tissue while preventing catching or damage. As discussed below, the sheath 350 can further receive a bone graft delivery instrument therethrough and guide delivery of bone graft onto a rasped surface. The open tip of the sheath can prevent inadvertent displacement of the bone graft material when the sheath is removed from the surgical site after bone graft is delivered.

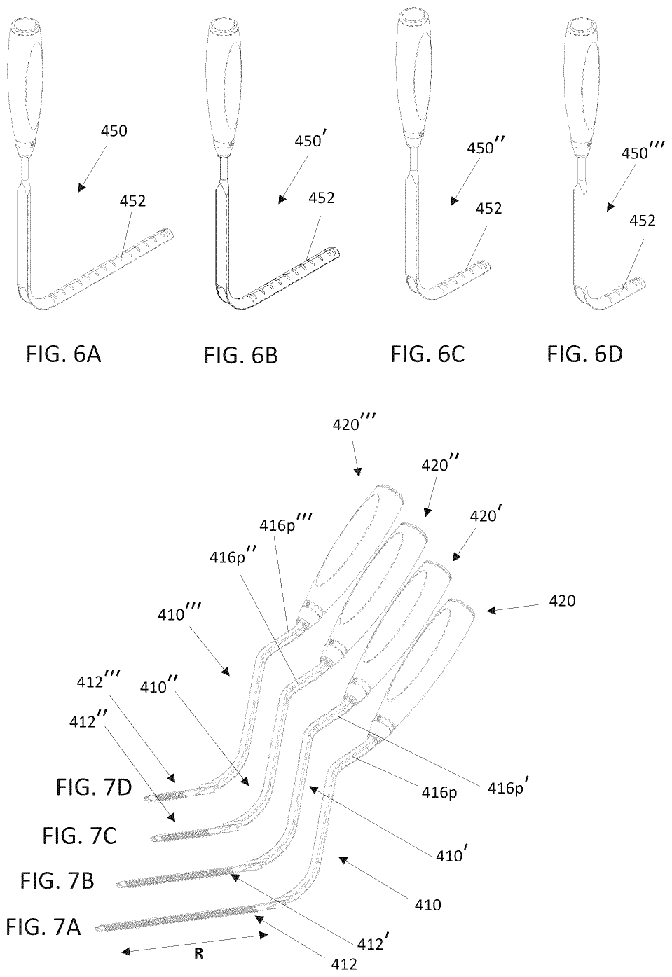

FIG. 6A-6D illustrate additional embodiments of sheaths 450, 450′, 450″, 450′″ that can be used with cutting or rasp instruments of the present disclosure. Various sheaths can be provided with distal portions of various length. The top surface of the distal portion of the sheath can include markings 452 indicating length measurements. In some procedures, the bone can be rasped prior to implanting a spinal rod, and the length markings 452 on the sheath can assist in measuring the length of the spinal rod needed to extend along the vertebrae.

Similarly, various cutting or rasp instruments 410, 410′, 410″, 410′″ illustrated in FIG. 7A-7D having distal portions 412, 412′, 412″, 412′″ of various lengths can be provided to nest within the sheaths 450, 450′, 450″, 450′″. During spinal fusion surgery, two or more levels of vertebrae along the length of the spine can be fused together using bone graft. In procedures requiring fusion of only two levels, the sheath 450 of FIG. 6D and the rasp 410′″ of FIG. 7D can be provided to rasp and guide bone graft delivery along the length of the two vertebrae. In other procedures requiring the fusion of more levels of vertebrae, the sheath 450 of FIG. 6A and the rasp 410 of FIG. 7A can be used to rasp and guide bone graft delivery along the length of multiple vertebrae. Additionally, as shown in FIG. 7A-7D, the proximal end of the rasp shaft 416p, 416p′, 416p″, 416p′″ can be angled to position a handle 420, 420′, 420″, 420′″ coupled thereto at an angle relative the longitudinal axis of a more central portion of the elongate shaft. The angled proximal end and handle can provide better ergonomics as a user moves the rasp instrument in a back and forth motion along a longitudinal axis of a distal portion of the instrument 410 to decorticate the bone surface, as shown by arrow R in FIG. 7A.

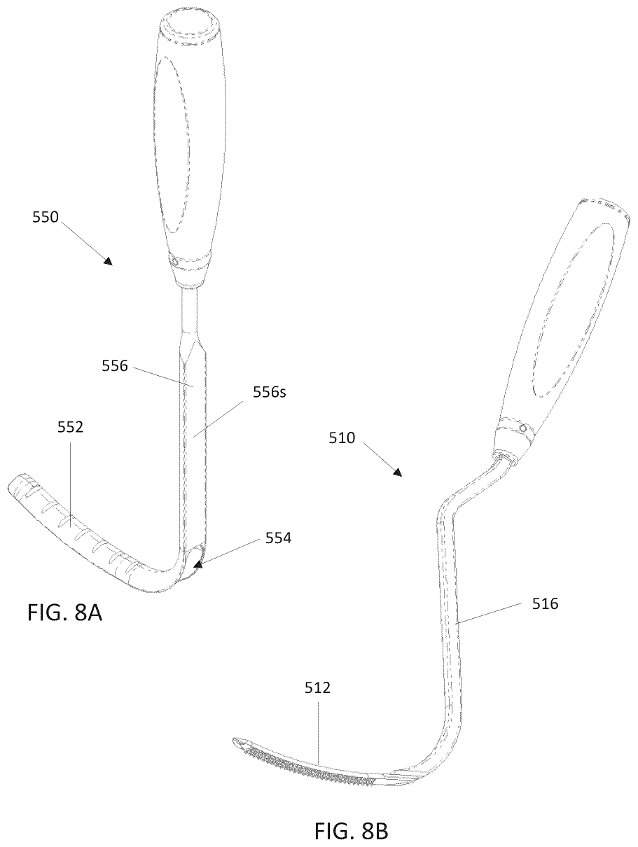

Further, in some embodiments the distal portion of the sheath and the distal portion of the rasp instrument received therein can be curved to match a curvature of the spine for improved contact with the bony anatomy. For example and as shown in FIG. 8A (illustrating sheath 550) and FIG. 8B (illustrating instrument 510), the distal portions 552, 512 can curve upwards for lordotic spinal curvatures. In some embodiments, distal portions 552, 512 can curve downwards for kyphotic spinal curvatures. As discussed above, the channel 554 of the sheath 550 can extend through at least a portion of the elongate body of the sheath. In some embodiments, the channel 554 can extend along the entire length to allow nesting of a proximal portion 516 of the rasp instrument 510 within the proximal portion 556 of the sheath 550. In the embodiment illustrated in FIG. 8A, the channel 554 extends through only the distal portion 552 of the sheath 550 such that the distal portion 512 of the rasp instrument 510 is nested within the distal portion 552 of the sheath 550 and the proximal portion 516 of the rasp instrument is positioned parallel to the proximal portion 556 of the sheath. The proximal portion 556 of the sheath can include a flat surface 556s to align against the proximal portion 516 of the rasp instrument.

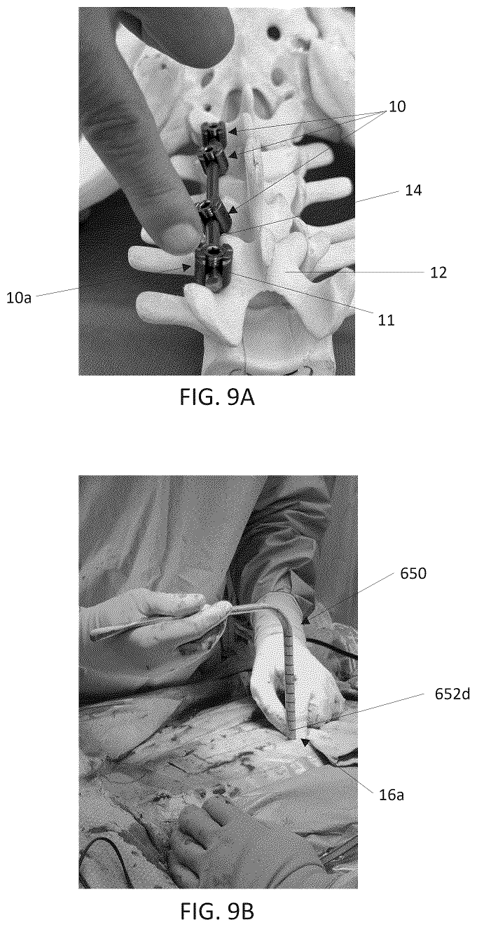

FIG. 9A-13B illustrate an example method of cutting or rasping bone surface and delivering bone graft using a system in accordance with the present disclosure. Prior to rasping bone, pedicle screws, implants, rods, or other components typically used in spinal surgery can be implanted into the bone, although in some procedures, the bone can be rasped prior to implanting. As previously discussed, the procedure can include fusion of two or more vertebrae 12. In the illustrated example, bone screws 10 are implanted into four vertebrae 12 via a minimally invasive procedure through small incisions posterior to the desired locations for each screw 10. A spinal rod 14 can be secured within the heads 11 of each screw 10, as shown in FIG. 9A. A surgeon can palpate through the proximal-most incision 16a to locate the proximal-most pedicle screw 10a and insert the distal tip 652d of the sheath 650 through the proximal-most incision, as shown in FIG. 9B. The distal tip 652d of the sheath 650 can be introduced directly over the proximal implant 10a and advanced toward the spine until it contacts bone adjacent to the lateral aspect of the implant 10a.

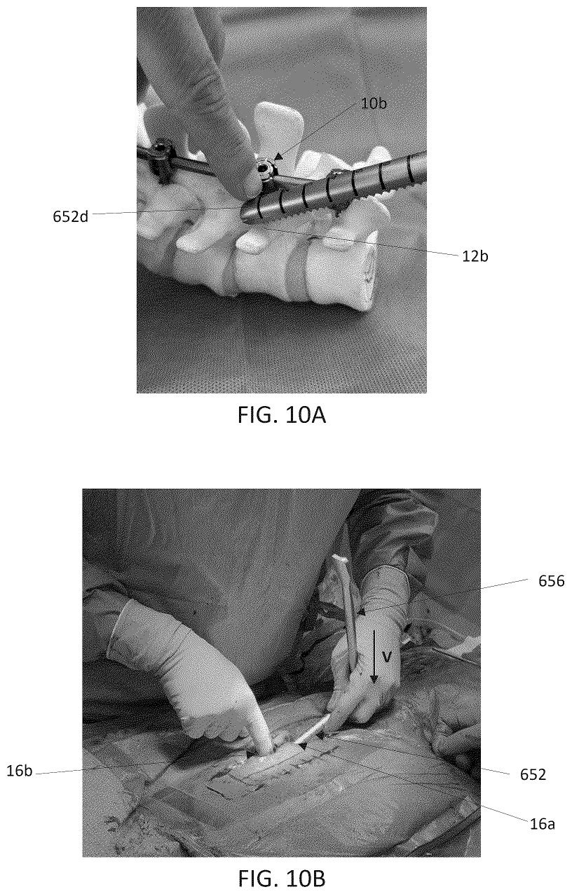

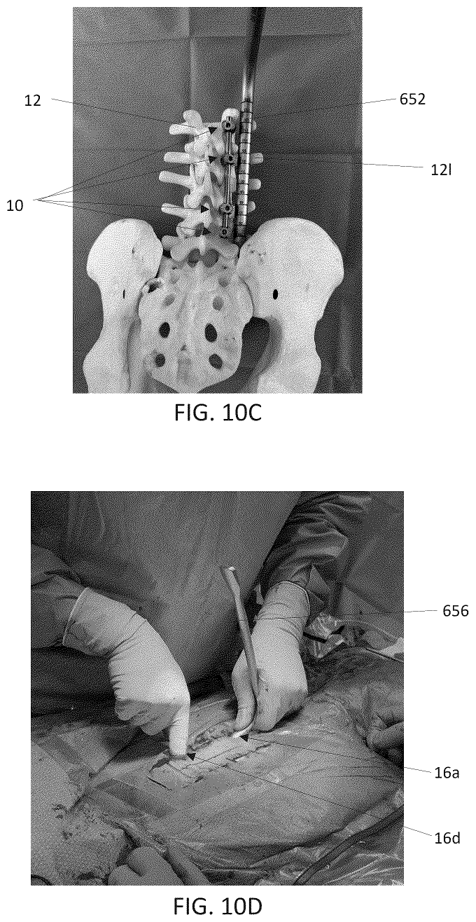

With reference to FIG. 10A-10D, the surgeon can then palpate the next implant 10b adjacent to the proximal-most implant 10a and move their finger laterally to locate bone surface 12b lateral to the adjacent implant 10b. The surgeon can then rotate the sheath 650 to bring a distal portion thereof into better alignment with the axis of the spine and advance the distal tip 652d of the sheath along the bone surface 12b until the distal tip 652d contacts the palpating finger lateral to the adjacent implant 10b. Ventral pressure in the direction V of FIG. 10B can be applied to maintain the distal portion 652 of the sheath against the bone surface under surrounding soft tissue while the sheath 650 is advanced along the vertebrae 12. The steps of palpating adjacent implants 10 and advancing the distal portion 652 of the sheath 650 can be repeated until the distal-most implant 10d is reached, thereby positioning the distal portion 652 of the sheath along the bone surface 12l lateral to the plurality of the implants 10, as shown in FIG. 10C.

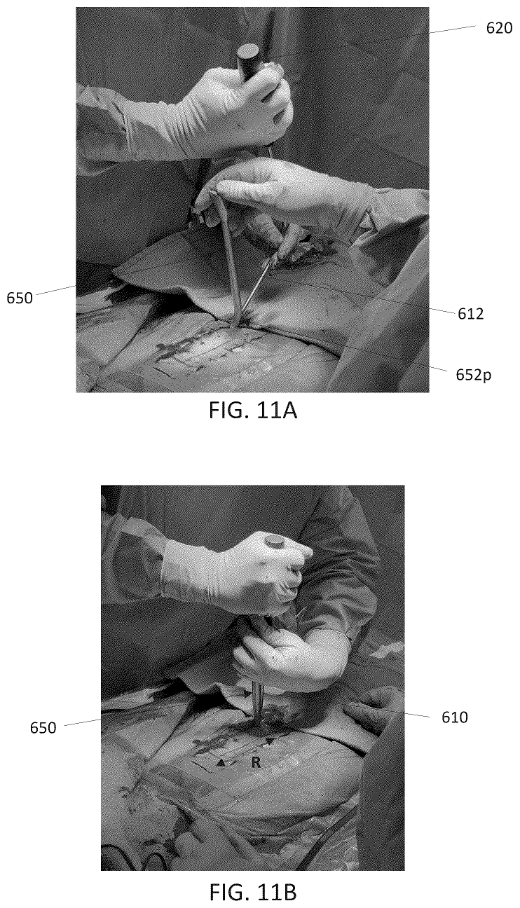

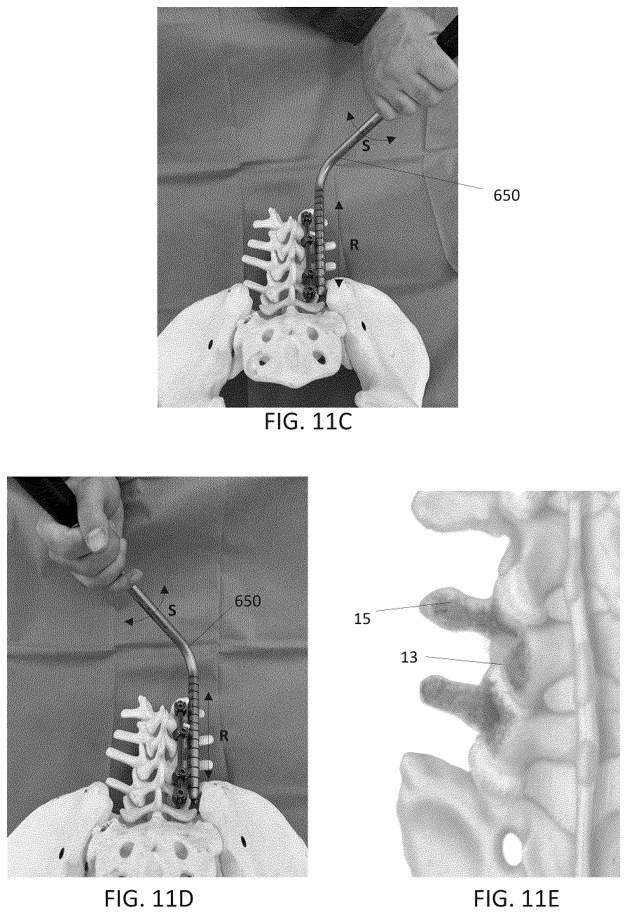

After the distal portion 652 of the sheath is positioned along the desired trajectory, the distal portion 612 of the cutting or rasp instrument 610 can be inserted into the sheath 650 by inserting the distal tip of the instrument 610 into the channel of the sheath at the proximal end 652p of the distal portion of the sheath, as shown in FIGS. 11A and 11B. While stabilizing the sheath 650, the instrument 610 can be manipulated in the cephalad and caudal directions to decorticate the underlying bone surface, as shown by arrow R in FIG. 11B. Additionally and as shown in FIGS. 11C and 11D, the instrument 610 and sheath 650 can be angled in the lateral or medial directions, as shown by arrow S in FIG. 11C. This movement of the rasp 610 within the sheath 650, along with the cephalad and caudal movement shown by arrow R in FIG. 11D, can be repeated to decorticate the vertebral bone. This can include decortication of the dorsal aspect of the inferior articular processes 13 and the dorsal aspect of the transverse processes 15. As shown in FIG. 11E.

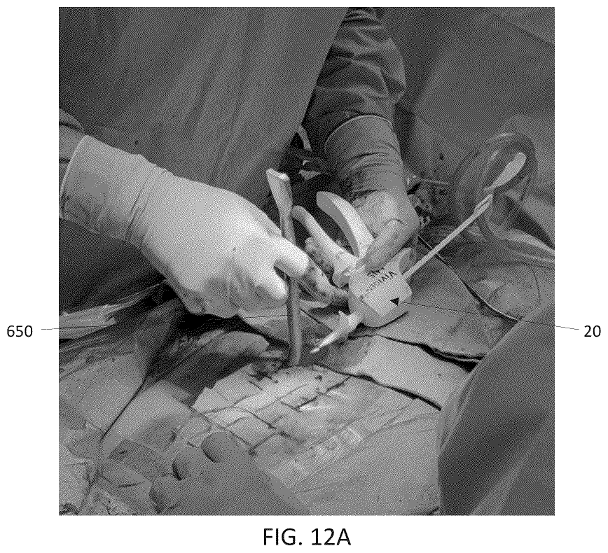

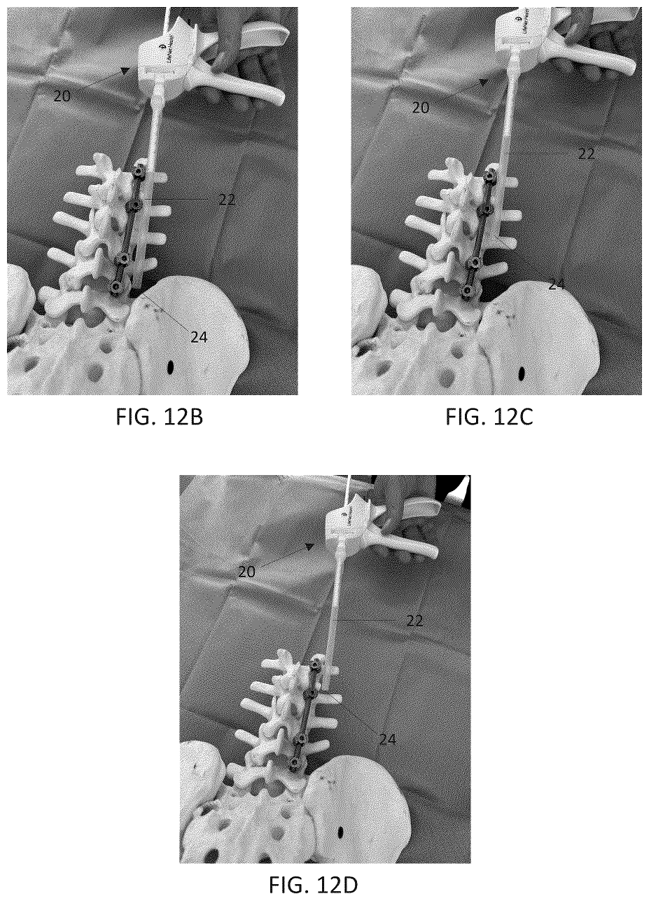

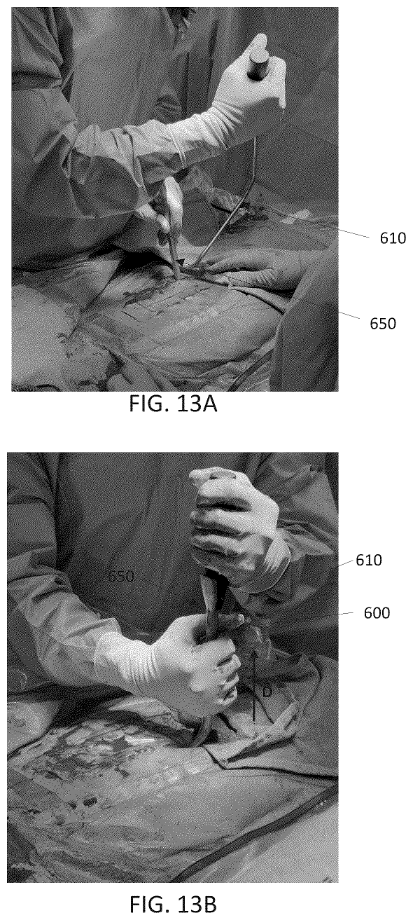

Following decortication of the bone surfaces, the rasp instrument 610 can be removed from the sheath 650 and surgical site while the position of the sheath 650 is maintained. As shown in FIG. 12A, a bone cement delivery instrument 20 can be inserted into the sheath channel, similar to the insertion of the rasp, to deliver bone graft material to the rasped surfaces. In some embodiments, the instrument 20 can be inserting in a manner that positions it adjacent to or proximate to the sheath 650. For example, the instrument 610 can be positioned below the sheath 650 such that it is disposed between the bone and the sheath 650. In the embodiment illustrated in FIG. 12A-12D, the bone cement delivery instrument 20 has an elongate cannula 22 sized to pass through the sheath channel with an opening 24 at the distal tip. The distal tip 24 of the cannula 22 can be advanced through the channel to the distal-most location of the distal tip 652d of the sheath and bone graft material can be slowly dispensed from the distal opening 24 while pulling the cannula 22 back through the sheath channel. In some embodiments, the rasp instrument 610 can be inserted back into the sheath 650 until the distal tip of the instrument 610 contacts the bone graft material to assist in maintaining the placement of the bone graft material while the system 600 is removed from the surgical site. In some embodiments, the sheath 650 and optionally rasp instrument 610 can be retracted away from the spine in the dorsal direction D shown in FIG. 13B to separate the distal portion of the sheath from the bone graft material. To fully remove the system from the surgical site, the surgeon can use an arcing retraction motion to extract the system 600 from the surgical site through the incision 16a, essentially reversing the motion utilized to insert the sheath 650. This can include retraction in a caudal direction if insertion proceeded in a cephalad direction, though the reverse is also possible.

As noted above In some embodiments, the above-described process of preparing bone can be performed using an alternative order of operations. For example, in some embodiments the preparation can be performed prior to implantation of any bone screw 10 or other implant. That is, the sheath 650 and instrument 610 can be utilized to prepare bone as described herein through one or more incisions prior to implanting one or more bone screws.

Various devices and methods disclosed herein can be used in minimally-invasive surgery and/or open surgery. While various devices and methods disclosed herein are generally described in the context of surgery on a human patient, the methods and devices disclosed herein can be used in any of a variety of surgical procedures with any human or animal subject, or in non-surgical procedures.

Various devices disclosed herein can be constructed from any of a variety of known materials. Example materials include those that are suitable for use in surgical applications, including metals such as stainless steel, titanium, titanium nitride, aluminum titanium nitride, nickel, cobalt, chrome, cobalt-chromium, aluminum, or alloys and combinations thereof, polymers such as Radel PPSU (polyphenylsulfone), PEEK (polyether ether ketone), ceramics, carbon fiber, and so forth. The various components of the devices disclosed herein can be rigid or flexible. In addition, one or more of the components or devices disclosed herein can be formed as monolithic or unitary structures, e.g., formed from a single continuous material, or can be formed from separate components coupled together in a variety of manners that either facilitate or discourage subsequent separation. One or more components or portions of the device can be formed from a radiopaque material to facilitate visualization under fluoroscopy and other imaging techniques, or from a radiolucent material so as not to interfere with visualization of other structures. Example radiolucent materials include carbon fiber and high-strength polymers. Further, various methods of manufacturing can be utilized, including 3D printing or other additive manufacturing techniques, as well as more conventional manufacturing techniques, including molding, stamping, casting, machining, etc.

Various devices or components disclosed herein can be designed to be disposed of after a single use, or they can be designed to be used multiple times. In either case, however, various devices or components can be reconditioned for reuse after at least one use. Reconditioning can include any combination of the steps of disassembly, followed by cleaning or replacement of particular pieces, and subsequent reassembly. In particular, a device or component can be disassembled, and any number of the particular pieces or parts thereof can be selectively replaced or removed in any combination. Upon cleaning and/or replacement of particular parts, the device or component can be reassembled for subsequent use either at a reconditioning facility, or by a surgical team immediately prior to a surgical procedure. Reconditioning of a device or component can utilize a variety of techniques for disassembly, cleaning/replacement, and reassembly. Use of such techniques, and the resulting reconditioned device or component, are within the scope of the present disclosure.

Various devices or components described herein can be processed before use in a surgical procedure. For example, a new or used device or component can be obtained and, if necessary, cleaned. The device or component can be sterilized. In one sterilization technique, the device or component can be placed in a closed and sealed container, such as a plastic or TYVEK bag. The container and its contents can be placed in a field of radiation that can penetrate the container, such as gamma radiation, x-rays, or high-energy electrons. The radiation can kill bacteria on the device or component and in the container. The sterilized device or component can be stored in the sterile container. The sealed container can keep the device or component sterile until it is opened in the medical facility. Other forms of sterilization are also possible, including beta or other forms of radiation, ethylene oxide, steam, or a liquid bath (e.g., cold soak). Certain forms of sterilization may be better suited to use with different devices or components, or portions thereof, due to the materials utilized, the presence of electrical components, etc.

In this disclosure, articles “a” and “an” are used to refer to one or to more than one (i.e., at least one) of the grammatical object of the article. By way of example, “an element” means at least one element and can include more than one element. The term “about” is used to provide flexibility to a numerical range endpoint by providing that a given value may be “slightly above” or “slightly below” the endpoint without affecting the desired result. The use herein of the terms “including,” “comprising,” or “having,” and variations thereof, is meant to encompass the elements listed thereafter and equivalents thereof, as well as additional elements. As used herein, “and/or” refers to and encompasses any and all possible combinations of one or more of the associated listed items, as well as the lack of combinations where interpreted in the alternative (“or”). Further, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B,” “one or more of A and B,” and “A and/or B” are each intended to mean “A alone, B alone, or A and B together.” A similar interpretation is also intended for lists including three or more items. For example, the phrases “at least one of A, B, and C,” “one or more of A, B, and C,” and “A, B, and/or C” are each intended to mean “A alone, B alone, C alone, A and B together, A and C together, B and C together, or A and B and C together.” In addition, use of the term “based on,” is intended to mean, “based at least in part on,” such that an un-recited feature or element is also permissible.

To the extent that linear, circular, or other dimensions are used in the description of the disclosed devices and methods, such dimensions are not intended to limit the types of shapes that can be used in conjunction with such devices and methods. Equivalents to such dimensions can be determined for different geometric shapes, etc. Further, like-numbered components of the embodiments can generally have similar features. Still further, sizes and shapes of the devices, and the components thereof, can depend at least on the anatomy of the subject in which the devices will be used, the size and shape of objects with which the devices will be used, and the methods and procedures in which the devices will be used. To the extent that features, objects, steps, or the like are described as being “first,” “second,” third,” etc., and/or “lower,” “upper,” “middle,” etc., such numerical and/or location ordering/identification is generally arbitrary, and thus such numbering can be interchangeable unless otherwise indicated.

The figures provided herein are not necessarily to scale. Further, to the extent arrows are used to describe a direction of movement, these arrows are illustrative and in no way limit the direction that the respective component can or should be moved. Other movements and directions may be possible to create the desired result in view of the present disclosure. To the extent that directional terms, such as “proximal” and “distal,” are used in the description of the disclosed devices and methods, such terms are being measured with respect to anatomical position of a patient, though these terms may be relative and the meaning of these terms can be interchanged without changing the scope of the present disclosure. Likewise, in some embodiments movement of one component may be described with respect to another, but other movements may also be possible and/or may be described using different frames of reference.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Further features and advantages based on the above-described embodiments are possible and within the scope of the present disclosure. Accordingly, the disclosure is not to be limited by what has been particularly shown and described. All publications and references cited herein are expressly incorporated herein by reference in their entirety, except for any definitions, subject matter disclaimers, or disavowals, and except to the extent that the incorporated material is inconsistent with the express disclosure herein, in which case the language in this disclosure controls.

Examples of the above-described embodiments can include the following:
1. A method of preparing a bone for surgery, comprising:
   inserting a delivery sheath through a first incision formed proximate to a first pedicle screw disposed in bone;
   advancing the delivery sheath toward one or more additional pedicle screws disposed in bone;
   inserting an instrument into the delivery sheath through the first incision, the instrument having an elongate shaft with a cutting surface formed thereon;
   passing the instrument over the bone in which the one or more additional pedicle screws are disposed to decorticate the bone with the cutting surface;
   removing the instrument from the sheath;
   passing at least one of a bone graft or a biologic material through the sheath to deliver the at least one of the bone graft or the biologic material to a surface of the bone exposed by the decorticating of the bone.
2. The method of example 1, further comprising:
   palpating a head of a second pedicle screw of the one or more additional pedicle screws using a finger through a second incision formed proximate to the second pedicle screw,
   wherein advancing the delivery sheath toward the second pedicle screw disposed in bone further comprises contacting the finger at the head of the second pedicle screw that was palpated.
3. The method of any of examples 1 to 2, wherein the one or more additional pedicle screws comprises a plurality of additional pedicle screws.
4. The method of any of examples 1 to 3, wherein advancing the delivery sheath toward one or more additional pedicle screws disposed in bone further comprises advancing the delivery sheath to a distal-most pedicle screw of the one or more additional pedicle screws.
5. The method of any of examples 1 to 4, wherein passing the instrument over the bone in which the one or more additional pedicle screws are disposed to decorticate the bone with the cutting surface further comprises angling the instrument in each of a cephalad direction and a caudal direction.
6. The method of any of examples 1 to 5, wherein passing the instrument over the bone in which the one or more additional pedicle screws are disposed to decorticate the bone with the cutting surface further comprises decorticating a dorsal aspect of an inferior articular process.
7. The method of any of examples 1 to 6, wherein passing the instrument over the bone in which the one or more additional pedicle screws are disposed to decorticate the bone with the cutting surface further comprises decorticating a dorsal aspect of a transverse process.
8. The method of any of examples 1 to 7, wherein passing at least one of a bone graft or a biologic material through the sheath further comprises inserting a delivery instrument into the sheath to deliver the at least one of the bone graft or the biologic material to the surface of the bone exposed by the decorticating of the bone.
9. The method of any of examples 1 to 8, wherein passing at least one of a bone graft or a biologic material through the sheath comprises passing a bone graft through the sheath and, after the bone graft is delivered to the surface of the bone exposed by the decorticating of the bone, the method further comprises reinserting the instrument into the sheath and contacting the delivered bone graft with the instrument.
10. The method of example 9, further comprising removing the sheath through the first incision so it is no longer disposed at the bone while keeping the instrument in contact with the bone graft.
11. The method of any of examples 1 to 10, wherein a distal portion of the sheath extends at an angle relative to a proximal portion of the sheath and the proximal portion extends out from the first incision after inserting the delivery sheath through the first incision.
12. The method of any of examples 1 to 11, wherein a distal portion of the instrument extends at an oblique angle relative to a proximal portion of the instrument and the proximal portion extends out from the first incision after inserting the instrument into the delivery sheath.
13. The method of any of examples 1 to 12, wherein a longitudinal axis of a handle of the delivery sheath forms an oblique angle with a longitudinal axis of a handle of the instrument when the instrument is inserted into the delivery sheath.
14. The method of any of examples 1 to 13, wherein passing the instrument over the bone in which the one or more additional pedicle screws are disposed to decorticate the bone with the cutting surface further comprises angling at least one of the instrument and the sheath in one of the medial or lateral directions and translating the instrument relative to the sheath along a longitudinal axis of a distal portion thereof.
15. The method of any of examples 1 to 14, further comprising adjusting a position of a handle of the instrument relative to the elongate shaft of the instrument.
16. A surgical system, comprising:
   a cutting instrument that includes a handle and an elongate shaft, the elongate shaft having a proximal portion and a distal portion, the distal portion being disposed at an angle with respect to the proximal portion and the distal portion having a cutting surface formed thereon; and
   a sheath that includes a handle and an elongate receiving portion that includes a channel extending from a proximal portion of the elongate receiving portion to a distal portion of the elongate receiving portion, the distal portion of the elongate receiving portion being disposed at an angle with respect to the proximal portion of the elongate receiving portion, and the channel being configured to receive the elongate shaft of the cutting instrument such that the cutting surface of the cutting instrument is exposed to be able to decorticate bone.
17. The system of example 16, wherein the channel of the sheath is open along a length thereof.
18. The system of any of examples 16 to 17, wherein a distal-most tip of the elongate receiving portion of the sheath has an opening into the channel.
19. The system of any of examples 16 to 18, wherein a distal-most tip of the elongate shaft of the cutting instrument is configured to extend from the opening of the sheath when the distal portion of the elongate shaft is received within the distal portion of the elongate receiving portion of the sheath.
20. The system of example 19, wherein the distal-most tip of the elongate shaft is bulleted.
21. The system of any of examples 16 to 20, wherein the distal portion of the elongate shaft and the distal portion of the elongate receiving portion of the sheath are straight, each having a convex upper surface and a flat lower surface.
22. The system of any of examples 16 to 21, wherein the distal portion of the elongate shaft and the distal portion of the elongate receiving portion of the sheath are curved along a longitudinal axis thereof.
23. The system of any of examples 16 to 22, wherein the distal portion of the elongate receiving portion of the sheath extends at a perpendicular angle relative to the proximal portion of the elongate receiving portion of the sheath.
24. The system of any of examples 16 to 23, wherein the proximal portion of the elongate shaft of the cutting instrument extends at an oblique angle relative to the distal portion of the elongate shaft of the cutting instrument.
25. The system of any of examples 16 to 24, wherein the handle is fixed relative to the elongate shaft and a longitudinal axis of the handle is coaxial with a longitudinal axis of the proximal portion of the elongate shaft.
26. The system of any of examples 16 to 25, wherein the handle is fixed relative to the elongate shaft and a longitudinal axis of the handle forms an oblique angle with a longitudinal axis of the proximal portion of the elongate shaft.
27. The system of any of examples 16 to 26, wherein a position of the handle can be adjusted relative to the elongate shaft.
28. The system of any of examples 16 to 27, wherein the cutting surface includes a plurality of teeth.
29. The system of any of examples 16 to 28, wherein the distal portion of the elongate receiving portion of the sheath is configured to receive an instrument for depositing bone graft.

What is claimed is:

1. A method of preparing a bone for surgery, comprising:
inserting a delivery sheath through a first incision formed proximate to a first pedicle screw disposed in bone;
advancing the delivery sheath toward one or more additional pedicle screws disposed in bone;
inserting an instrument into the delivery sheath through the first incision, the instrument having an elongate shaft with a cutting surface formed thereon;
passing the instrument over the bone in which the one or more additional pedicle screws are disposed to decorticate the bone with the cutting surface;
removing the instrument from the sheath;
passing at least one of a bone graft or a biologic material through the sheath to deliver the at least one of the bone graft or the biologic material to a surface of the bone exposed by the decorticating of the bone.

2. The method of claim 1, further comprising:
palpating a head of a second pedicle screw of the one or more additional pedicle screws using a finger through a second incision formed proximate to the second pedicle screw,
wherein advancing the delivery sheath toward the second pedicle screw disposed in bone further comprises contacting the finger at the head of the second pedicle screw that was palpated.

3. The method of claim 1, wherein the one or more additional pedicle screws comprises a plurality of additional pedicle screws.

4. The method of claim 1, wherein advancing the delivery sheath toward one or more additional pedicle screws disposed in bone further comprises advancing the delivery sheath to a distal-most pedicle screw of the one or more additional pedicle screws.

5. The method of claim 1, wherein passing the instrument over the bone in which the one or more additional pedicle screws are disposed to decorticate the bone with the cutting surface further comprises angling the instrument in each of a cephalad direction and a caudal direction.

6. The method of claim 1, wherein passing the instrument over the bone in which the one or more additional pedicle screws are disposed to decorticate the bone with the cutting surface further comprises decorticating a dorsal aspect of an inferior articular process.

7. The method of claim 1, wherein passing the instrument over the bone in which the one or more additional pedicle screws are disposed to decorticate the bone with the cutting surface further comprises decorticating a dorsal aspect of a transverse process.

8. The method of claim 1, wherein passing at least one of a bone graft or a biologic material through the sheath further comprises inserting a delivery instrument into the sheath to deliver the at least one of the bone graft or the biologic material to the surface of the bone exposed by the decorticating of the bone.

9. The method of claim 1, wherein passing at least one of a bone graft or a biologic material through the sheath comprises passing a bone graft through the sheath and, after the bone graft is delivered to the surface of the bone exposed by the decorticating of the bone, the method further comprises reinserting the instrument into the sheath and contacting the delivered bone graft with the instrument.

10. The method of claim 9, further comprising removing the sheath through the first incision so it is no longer disposed at the bone while keeping the instrument in contact with the bone graft.

11. The method of claim 1, wherein a distal portion of the sheath extends at an angle relative to a proximal portion of the sheath and the proximal portion extends out from the first incision after inserting the delivery sheath through the first incision.

12. The method of claim 1, wherein a distal portion of the instrument extends at an oblique angle relative to a proximal portion of the instrument and the proximal portion extends out from the first incision after inserting the instrument into the delivery sheath.

13. The method of claim 1, wherein a longitudinal axis of a handle of the delivery sheath forms an oblique angle with a longitudinal axis of a handle of the instrument when the instrument is inserted into the delivery sheath.

14. The method of claim 1, wherein passing the instrument over the bone in which the one or more additional pedicle screws are disposed to decorticate the bone with the cutting surface further comprises angling at least one of the instrument and the sheath in one of the medial or lateral directions and translating the instrument relative to the sheath along a longitudinal axis of a distal portion thereof.

15. The method of claim 1, further comprising adjusting a position of a handle of the instrument relative to the elongate shaft of the instrument.
